(19) United States
(12) Patent Application Publication
(10) Pub. No.: US 2026/0138714 A1
(43) Pub. Date: May 21, 2026

(54) Apparatus and Process for Ship-Based Carbon Dioxide Capture for Fuel Production

(71) Applicant: Air Products and Chemicals, Inc., Allentown, PA (US)

(72) Inventors: Hallgeir Dalane Angel, Kristiansand (NO); Leo Richard Nemetz, Chesterfield, MO (US); Steffen Zahn, Pennsburg, PA (US)

(73) Assignee: Air Products and Chemicals, Inc., Allentown, PA (US)

(21) Appl. No.: 18/950,820

(22) Filed: Nov. 18, 2024

(51) Int. Cl. B63B 35/44; B63B 17/00; B63J 4/00; C10L 3/00

(52) U.S. Cl. CPC B63B 35/44; B63B 17/0027; B63J 4/00; C10L 3/00; B63B 2035/4486; C10L 2200/0254; C10L 2290/42; C10L 2290/567

(57) ABSTRACT

An apparatus and process for capturing carbon dioxide from exhaust output from at least one ship engine on the ship for storage of the carbon dioxide on the ship. The storage of the carbon dioxide on the ship can facilitate delivery of the captured carbon dioxide to a fuel production system at a port of destination or other port that the ship may arrive at to provide the captured carbon dioxide to the fuel production system. The fuel production system may then use the carbon dioxide to form a fuel (e.g. methane, methanol, etc.). The fuel that is produced can be provided to other ships at the port. The carbon capture system can utilize membranes, adsorption, and/or absorption for capturing the carbon dioxide. The carbon dioxide can be stored on the ship in at least one storage vessel of the ship.

### FIELD OF THE INVENTION

The present innovation relates to ships and processes and apparatuses for capturing carbon dioxide from engine emissions of a ship to store the carbon dioxide (CO2) for use in delivering the captured CO2 to a fuel production system for producing a fuel.

### BACKGROUND OF THE INVENTION

Ships that travel on waterways such as an ocean, sea, lake, and/or river, can have engines that combust a fuel to power operation of the ship. Exhausts from these engines can result in the emission of carbon dioxide (CO2). Examples of different type of ships can be found in U.S. Patent Application Publication No. 2018/0161719, and Korean Patent Application Publication No. KR20220099193 A1.

### SUMMARY OF THE INVENTION

CO2 is a greenhouse gas that is believed to contribute to climate change. Maritime shipping can contribute to CO2 emissions via the combustion of fuel to power operations of ships. Ships can be retrofitted to include on-board CO2 capture systems to allow existing maritime fleets to be used while reducing or eliminating CO2 emissions. The aspect of disposing of or sequestering the captured carbon dioxide can be problematic and difficult because sequestration is often tied to specific geographical locations due to the geology that is typically required for CO2 storage, while the emissions in maritime shipping are decentralized and mobile. However, such issues can be addressed by repurposing captured CO2 that is captured from ship emissions so that the captured CO2 can be temporarily stored onboard a ship and supplied periodically to a port fuel production system that can allow for a conversion of the stored CO2 to a useable fuel instead of having to sequester the stored CO2 in a geological structure or other location. This can create a recycle loop of carbon within the maritime shipping industry that can help limit CO2 emissions.

Carbon formed via combustion of fuel in maritime shipping applications can be recycled in a loop within the maritime industry to greatly minimize the amount of greenhouse gasses emitted by the industry. For example, the decentralized nature of maritime CO2 emissions can be accounted for by allowing for CO2 capture on each individual ship while also permitting the captured CO2 to be utilized in fuel production as a source of the fuel.

A carbon capture system (CCS) may be incorporated into existing ships regardless of fuel source (i.e. diesel, heavy fuel oil, liquid natural gas, methanol, etc.) and this can allow for captured exhaust CO2 to be converted to a useful fuel by providing the captured CO2 as a CO2 supply source for a fuel producer that may be located near a port. This type of approach can enable the continued use of existing maritime vessels while also helping the vessels meet the International Maritime Organization goal of greatly reducing CO2 emissions from the maritime shipping industry.

Carbon capture systems that may be utilized can include an on-board CCS for a maritime ship to mitigate CO2 emissions by capturing CO2 in engine exhaust before the engine exhaust is vented to the atmosphere. The CCS system can be membrane based, adsorption based, and/or absorption based. The CO2 that is captured can be stored on board the ship until the ship arrives at a port. At the port, the captured and stored CO2 can be output from the ship to a port fuel production system to form fuel (e.g. via reacting the stored CO2 with hydrogen to form methanol, methane, dimethyl ether (DME), etc.). The CO2 from multiple ships can be supplied to the port fuel production systems at different port locations so that the captured CO2 can be converted to a maritime fuel at those locations (e.g. liquid natural gas (LNG), methanol, etc. can be formed in which the conversion of CO2 may be chemical or biological etc. for the forming of the fuel). The hydrogen (H2) that can be supplied for the conversion of CO2 can be from a carbon-free source (e.g. electrolysis of water powered by renewable power sources, etc.). In other embodiments the hydrogen can be from a process that does not emit CO2 or emits a relatively small amount of CO2 (e.g. a blue hydrogen process involving carbon capture technology to avoid or limit CO2 emissions during the making of the hydrogen). In some embodiments, a membrane system or other type of CO2 and/or H2 recovery system can be used at the port fuel production system (PFPS) to purify the fuel product formed using the ship-captured CO2 to recover any unreacted H2 and CO2 from the produced fuel before that fuel is stored for distribution to other ships that may be docked at the port in which the port fuel production system is positioned.

Embodiments can be configured to facilitate CO2 capture to help reduce CO2 emissions from maritime vessel operation and use while also permitting the captured CO2 to be utilized in production of fuel. Embodiments can permit a significant reduction in CO2 emissions as well as permitting a decentralized carbon capture approach in which the captured CO2 is put to productive use instead of being sequestered. Further, embodiments can permit methane, methanol, DME, or other fuel to be formed using the captured CO2 and hydrogen (H2) produced via green or blue processes so that the formed fuel can minimize or eliminate carbon dioxide emissions. This is particularly true for embodiments in which the hydrogen utilized at a PFPS is green hydrogen formed via electrolysis of water that is powered via renewable power sources (e.g. solar power, hydraulic power, and/or wind power, etc.).

In a first aspect, an apparatus for ship-based carbon dioxide (CO2) capture is provided. The apparatus can include a ship having a CO2 capture system positioned to receive flue gas from an engine of the ship to remove CO2 from the flue gas. The CO2 capture system can be configured so that CO2 removed from the flue gas may be stored in at least one CO2 storage device of the ship that is in fluid communication with a CO2 capture device of the CO2 capture system of the ship. The CO2 storage device of the ship can be releaseably connectable to a CO2 delivery conduit connectable to the CO2 storage device to feed CO2 from the CO2 storage device of the ship to at least one CO2 storage tank of a port fuel production system (PFPS) positioned at a port in which the ship is dockable or a CO2 delivery vehicle positionable adjacent the ship to receive the CO2 from the CO2 storage device of the ship to the delivery vehicle for supplying the CO2 to the PFPS.

In some embodiments, the delivery vehicle can be a tanker vessel or other type of vehicle having a CO2 storage vessel for transporting the CO2 to the PFPS.

The CO2 storage device of the ship can be one or more vessels retained on the ship or in a hull of the ship. In some embodiments, there can be a valve or outlet that is releaseably connectable to a CO2 delivery conduit for providing the CO2 to the delivery vehicle or the CO2 storage tank of a PFPS, for example, and the valve or outlet can be positioned on an external side of a hull of the ship or on a deck of the ship.

In some embodiments, the CO2 capture system can be configured as an adsorption system, a pressure swing adsorption system, an adsorber system, or a membrane system. Other embodiments may utilize another type of CO2 capture mechanism or a combination of such mechanisms.

In some embodiments, the ship can be a type of vessel that travels on water (e.g. ocean, sea, lake, and/or river). The ship can be, for example, an ocean liner, a cargo ship, a cruise ship, a container ship, a barge, a yacht, an ocean freight ship, a freighter, a tanker, a dry bulk carrier, a reefer ship, a feeder ship, or other type of maritime vessel.

In a second aspect, the apparatus can include the PFPS and the PFPS can include a fuel production system configured to form methane, dimethyl ether (DME), or methanol from the CO2 provided by the CO2 storage device of the ship and hydrogen from at least one source of hydrogen. In some embodiments, the fuel production system can include a methanation reactor and/or a methanation and membrane system.

In at third aspect, the apparatus can include the CO2 delivery vehicle positionable adjacent the ship to receive the CO2 from the CO2 storage device of the ship to the delivery vehicle for supplying the CO2 to the PFPS. The CO2 delivery vehicle can be an intermediate device that collects the CO2 from the ship and then delivers that CO2 to a PFPS, for example. In such an embodiment, the CO2 may be obtained from the ship without the ship having to dock at a port.

In a fourth aspect, the PFPS can be included in the apparatus and the PFPS can include a fuel storage device that is positioned to receive the fuel formed via the fuel production system. The fuel storage device can be connectable to a source of fuel of the ship via a ship fueling feed conduit connectable between the source of fuel of the ship and the fuel storage device of the PFPS. In some embodiments, the fuel can include methane, dimethyl ether (DME), or methanol and/or the source of hydrogen can be a hydrogen pipeline, a hydrogen storage tank, hydrogen formed via electrolysis of water, and/or hydrogen made via a blue hydrogen production process. In some embodiments, the electrolysis of water can be powered by renewable power.

In a fifth aspect, the apparatus of the first aspect can include one or more features of the second aspect, third aspect, and/or fourth aspect. Embodiments of the apparatus can also include other features or elements. Examples of such features or elements can be appreciated from the exemplary embodiments discussed herein.

In a sixth aspect, a process for ship-based CO2 capture is provided. The process can include capturing CO2 from flue gas emitted by an engine of a ship during operation of the ship via a carbon capture system of the ship, storing the captured CO2 in at least one CO2 storage device of the ship that is in fluid communication with a CO2 capture device of the CO2 capture system of the ship for subsequent delivery of the CO2 stored in the CO2 storage device of the ship to (i) a CO2 delivery conduit connectable to the CO2 storage device to feed CO2 from the CO2 storage device of the ship to at least one CO2 storage tank of a port fuel production system (PFPS) positioned at a port in which the ship is dockable or (ii) a CO2 delivery vehicle positionable adjacent the ship to receive the captured CO2 from the CO2 storage device of the ship to the delivery vehicle for supplying the CO2 to at least one CO2 storage tank of the PFPS.

In some embodiments, the CO2 capture can occur while the ship moves on water (e.g. an ocean, a sea, a river, a lake, etc.). Embodiments of the process can utilize an embodiment of the apparatus described herein as well.

In a seventh aspect, the process can also include docking the ship at the port, connecting the CO2 storage device of the ship to the CO2 delivery conduit to feed the CO2 stored in the CO2 storage device of the ship to the at least one CO2 storage tank of the PFPS, and feeding the CO2 from the CO2 storage device of the ship to the at least one CO2 storage tank of the PFPS via the CO2 delivery conduit.

In some embodiments, the process can also include feeding the CO2 from the at least one CO2 storage tank of the PFPS to a fuel production system of the PFPS to form a fuel and feeding hydrogen from a source of hydrogen of the PFPS to the fuel production system to form the fuel. The process may also include feeding the formed fuel from the fuel production system to a fuel storage device of the PFPS and/or feeding the fuel stored in the fuel storage device of the PFPS to a source of fuel of the ship.

In some embodiments, the feeding of the fuel to the source of fuel of the ship can occur at a same time the feeding of the CO2 from the CO2 storage device of the ship to the at least one CO2 storage tank of the PFPS via the CO2 delivery conduit occurs.

In an eighth aspect, the fuel can include methane, dimethyl ether (DME), or methanol in some embodiments. The hydrogen that can be used to form the fuel can be from a source of hydrogen, which can include hydrogen from a hydrogen pipeline, hydrogen from a hydrogen storage tank, hydrogen formed via electrolysis of water, and/or hydrogen made via a blue hydrogen production process.

In a ninth aspect, embodiments of the process of the sixth aspect can include one or more features of the seventh aspect and/or the eighth aspect. Embodiments of the process can also utilize other features or elements. Examples of such features or elements can be appreciated from the exemplary embodiments discussed herein.

In a tenth aspect, a system for ship-based carbon dioxide (CO2) capture is provided. The system can include a ship having a CO2 capture system positioned to receive flue gas from an engine of the ship to remove CO2 from the flue gas. The CO2 capture system can be configured so that CO2 removed from the flue gas is storable in at least one CO2 storage device of the ship that is in fluid communication with a CO2 capture device of the CO2 capture system of the ship. The CO2 storage device of the ship can be releaseably connectable to a CO2 delivery conduit connectable to the CO2 storage device to feed CO2 from the CO2 storage device of the ship to at least one CO2 storage tank of a port fuel production system (PFPS) positioned at a port in which the ship is dockable. The PFPS can also have a source of hydrogen and a fuel production system that is fluidly connectable to the source of hydrogen and the at least one CO2 storage tank of the PFPS so that a fuel is producible via the fuel production system via use of the CO2 from the at least one CO2 storage tank and hydrogen from the source of hydrogen.

In some embodiments, the source of hydrogen can include hydrogen produced via electrolysis of water powered by renewable power or hydrogen produced via a blue hydrogen production process. The fuel that can be produced can be a fuel that is comprised of methane, dimethyl ether (DME), or methanol.

In some embodiments, the PFPS can have a fuel storage device connected to the fuel production system to receive and store the fuel output from the fuel production system. The fuel storage device of the PFPS can be fluidly connectable to a source of fuel of the ship via a ship fueling feed conduit connectable between the source of fuel of the ship and the fuel storage device of the PFPS.

Embodiments of the system can also utilize other features or elements. Examples of such other elements or features can be appreciated from the exemplary embodiments discussed herein.

It should be appreciated that embodiments of the process and apparatus can utilize various conduit arrangements and process control elements. The embodiments may utilize sensors (e.g., pressure sensors, temperature sensors, flow rate sensors, concentration sensors, etc.), controllers, valves, piping, and other process control elements. Some embodiments can utilize an automated process control system and/or a distributed control system (DCS), for example. Various different conduit arrangements and process control systems can be utilized to meet a particular set of design criteria.

Other details, objects, and advantages of the apparatus for ship-based carbon dioxide (CO2) capture for fuel production, a process for ship-based CO2 capture for fuel production, a fuel production system, and methods of making and using the same will become apparent as the following description of certain exemplary embodiments thereof proceeds.

### BRIEF DESCRIPTION OF THE DRAWINGS

Exemplary embodiments of an apparatus for a ship-based carbon dioxide (CO2) capture for fuel production, a process for a ship-based CO2 capture for fuel production, a fuel production system, and methods of making and using the same are shown in the drawings included herewith. It should be understood that like reference characters used in the drawings may identify like components.

FIG. 1 (also referred to as FIG. 1) is a block diagram schematically illustrating a first exemplary embodiment of a process for ship-based CO2 capture for use in fuel production. An exemplary embodiment of an apparatus 1 for ship-based CO2 capture is also illustrated in this Figure.

FIG. 2 (also referred to as FIG. 2) is a block diagram of a first exemplary implementation of the first exemplary embodiment of a carbon capture system (CCS) that can be utilized in the first exemplary embodiment of the apparatus 1 for ship-based CO2 capture. An exemplary embodiment of a process for ship-based CO2 capture is also illustrated in this Figure.

FIG. 3 (also referred to as FIG. 3) is a block diagram of a first exemplary embodiment of a port fuel production system (PFPS) that can be utilized in the first exemplary embodiment of a process for ship-based CO2 capture for use in fuel production and an exemplary embodiment of an apparatus 1 for ship-based CO2 capture for use in fuel production.

FIG. 4 (also referred to as FIG. 4) is a flow chart of an exemplary embodiment of a process for ship-based CO2 capture for use in fuel production.

Reference numerals utilized in the drawings include:
1 apparatus for ship-based CO2 capture;
2 ship
3 fuel source (FS) for the ship 2 (e.g. fuel tank, etc.);
4 engine;
5 CCS feed conduit;
6 carbon capture system (CCS);
6cd CO2 carbon capture device;
6f CO2 feed stream;
6o vent treatment device;
6p CO2 purification device;
6s CO2 storage device;
6v impurity stream;
7 vent conduit;
21 port fuel production system (PFPS);
21d fuel storage device for the PFPS;
21f ship fueling feed conduit;
21h source of hydrogen (H2);
21p fuel production system;
21s CO2 delivery conduit;
21v CO2 storage tank for the PFPS;
23 hydrogen (H2) feed;
S1 first step;
S2 second step; and
S3 third step.

### DETAILED DESCRIPTION OF THE INVENTION

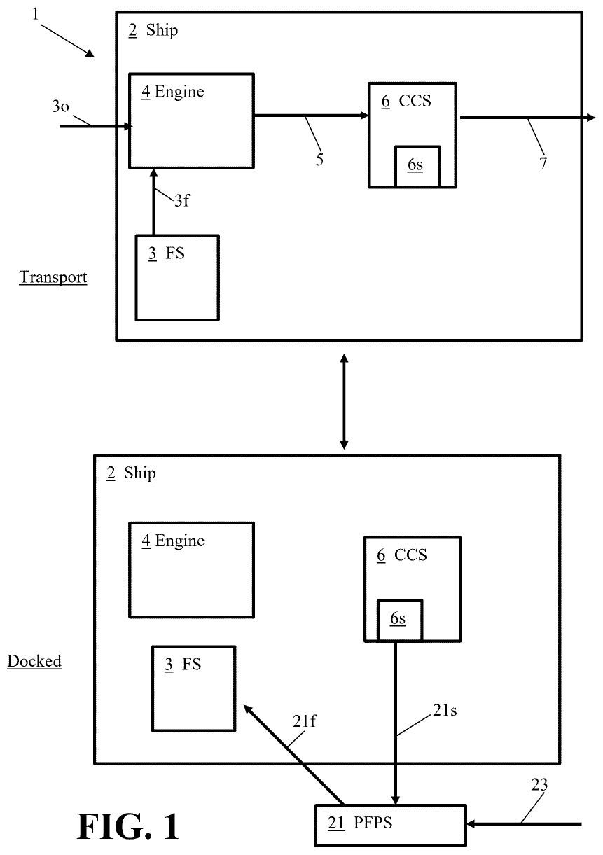

Referring to FIGS. 1-3, an apparatus 1 for ship-based CO2 capture can include a carbon capture system (CCS) 6 that is positioned on a ship 2. The ship 2 can be a maritime vessel. Examples of a ship 2 can include a cruise ship, a container ship, a barge, a yacht, a cargo ship, an ocean liner, an ocean freight ship, a freighter, a tanker, a dry bulk carrier, a reefer ship, a feeder ship, or other type of maritime vessel. The ship 2 can travel through sea water (e.g. a sea and/or at least one ocean, etc.) and/or fresh water (e.g. a lake and/or a river) and the CCS 6 can be positioned and configured to capture carbon dioxide (CO2) from flue gas formed via an engine 4 of the ship 2 combusting a fuel (e.g. liquid natural gas, methanol, diesel, etc.) to power propulsion of the ship and/or operation of the ship 2.

For example, the ship 2 can have at least one engine 4 that powers operation of the ship and/or the propulsion system of the ship 2 so the ship can move in water to travel from a port of origin to a port of destination. The engine 4 can include one or more furnaces or boilers or other type of engine that can combust fuel from a fuel source (FS) and oxidant received from a source of oxidant (e.g. air, etc.). For instance, the fuel source 3 can be at least one fuel tank of the ship that is fluidly connected to the engine 6 via a fuel feed conduit 3f that is positioned between the fuel source 3 and the engine 4. A source of oxidant (e.g. air, oxygen or an oxygen-containing component, etc.) can be fed into the engine via an oxidant feed conduit 3o that can be positioned between a source of oxidant and the engine 4. In some embodiments, the oxidant feed conduit 3o can include a fan or compressor that can receive air from the atmosphere and feed the air to the engine 4 as an oxidant. In some embodiments, the air may be purified prior to being fed to the engine 4 via at least one air filter element or other air purification mechanism that can be positioned upstream of the engine 4 and in fluid communication with the oxidant feed conduit 3o (e.g. to remove particulates and/or salt from the air, etc.).

The CCS 6 can be positioned downstream of the engine 4 so the CCS can receive flue gas formed from the combustion of the fuel from the engine 4. The flue gas output from the engine 4 can be fed to the CCS 6 via a CCS feed conduit 5 positioned between the engine 4 and the CCS 6. The CCS 6 can remove CO2 from the flue gas via an adsorption system, an absorption system, and/or a membrane system to output a ventable emission stream via a vent conduit 7 that is fluidly connected to the CCS 6 for passing the ventable emission stream output from the CCS 6 off the ship 2 and into the atmosphere. The ventable emissions stream can be the treated flue gas output from the CCS 6 that has no CO2 therein or a significant reduction in CO2 (e.g. includes water vapor, carbon monoxide, etc.).

In some embodiments, the engine 4 and the fuel source 3 can be positioned in a hull of the ship 2. A pump can be provided to feed the fuel from the fuel source 3 to at least one combustion chamber of the engine 4 in some embodiments. A fan or compressor can also be positioned on the ship to feed the oxidant (e.g. air, oxygen or an oxygen-containing component) to the engine 4 for combustion of the fuel.

The CCS 6 can include at least one CO2 capture device 6cd. In some embodiments, the CO2 capture device 6cd can include one or more adsorbers of a pressure swing adsorption (PSA) system, a temperature swing adsorption (TSA) system, or other type of adsorption system. In other embodiments, the CO2 capture device can include a CO2 capture membrane system configured to remove CO2 from the flue gas passed through one or more membranes of the membrane system. In yet other embodiments, the CO2 capture device 6cd can include an absorption system configured to absorb CO2 from the flue gas passed through absorption media of the absorption system. In other embodiments the CCS system can be a combination of two or more CO2 capture technologies outlined above. The treated flue gas output from the CCS 6 can have significantly less CO2 in the flue gas. In some embodiments, the CO2 reduction can be between a 100% reduction of CO2 in the flue gas (e.g. removal of all CO2 from the flue gas) to a removal of at least 30% of the CO2 from the flue gas. In some embodiments, the CCS 6 can be configured to remove between 50% and 99% or between 55% and 95% of the CO2 in the flue gas output from the engine 4, for example.

The CO2 capture device 6cd can be connected to the CCS feed conduit 5 so the CO2 capture device 6cd of the CCS 6 is positioned between the engine 4 and the vent conduit 7 to treat flue gas output from the engine 4. The flue gas can be passed through the CO2 capture device 6cd before the treated fluid is fed to the vent conduit 7 for venting to atmosphere.

The CO2 capture device 6cd can also be fluidly connected to a CO2 storage feed conduit 6f positioned between a CO2 storage device 6s and the CO2 capture device 6. A CO2 purification device 6p can optionally be positioned between the CO2 storage device 6s and the CO2 capture device 6 for purifying the CO2 to be fed to the CO2 storage device 6s.

The CO2 storage device 6s can be at least one tank or other vessel that is configured to store the CO2 output from the CO2 capture device 6cd. The stored CO2 can be compressed and/or cooled to liquify the CO2 for storage in the storage device(s) 6s in some embodiments. The CO2 storage device(s) 6s can be positioned in a hull of the ship 2 or other location on the ship 2.

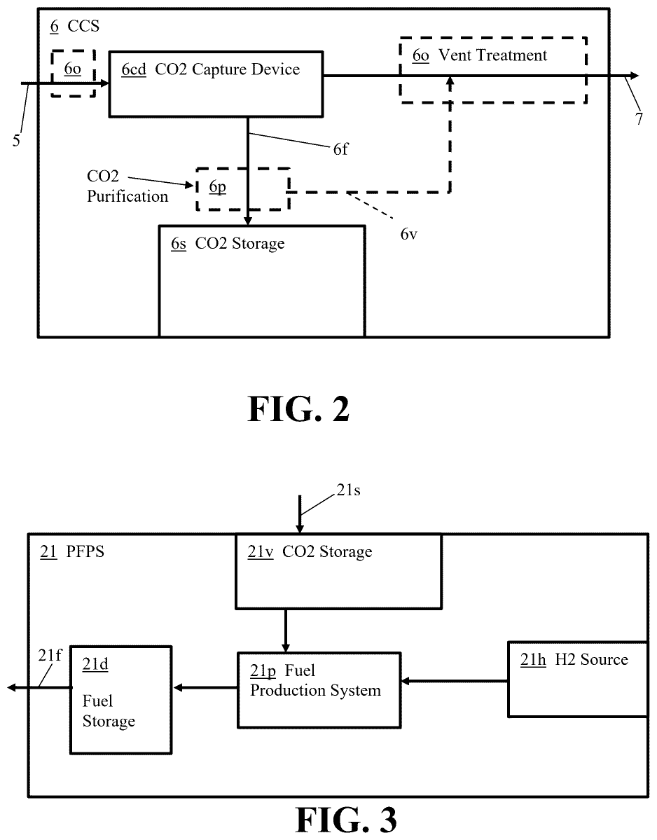

In embodiments that may utilize a purification device 6p (shown in broken line in FIG. 2), the CO2 purification device 6p can be connected to a vent treatment device 6o and/or a vent conduit 7 so that impurities removed from the CO2 stream passed through the CO2 storage feed conduit 6f can be vented to atmosphere. An impurity conduit 6v can be positioned between the CO2 purification device 6p and the vent conduit 7 for passing a stream of impurities removed from the CO2 being fed to the CO2 storage device 6s to the vent conduit 7.

In some embodiments, the CCS 6 can also include at least one vent treatment device 6o (shown in broken line in FIG. 2). A vent treatment device 6o can be configured to remove pre-selected constituents from the flue gas and/or cool the flue gas before it is output from the CCS 6 as a ventable stream. For example, the vent treatment device 6o can include at least one heat exchanger for utilizing heat from the ventable stream prior to it being emitted and/or a filtration device for removal of some constituents or particulates of the flue gas. As another example, a vent treatment device 6o can be positioned upstream of the CO2 capture device for removal of particulates or other elements from the flue gas prior to the flue gas being fed to the CO2 capture device and/or downstream of the carbon capture device 6cd to remove particulates or other elements from the flue gas to from the ventable stream for outputting the ventable stream to atmosphere via the vent conduit 7.

The ship 2 and the CO2 storage device(s) 6s can be positioned and configured so that CO2 from within the CO2 storage device(s) can be releasably coupled to a CO2 delivery conduit 21s for supplying CO2 from the CO2 storage device(s) 6s that are on the ship 2 to a port fuel production system CO2 storage device 21v or a CO2 delivery vehicle. The port fuel production system CO2 storage device 21v can include at least one tank for storage of the CO2 received from the ship 2 when the ship is docked at port, for example. The CO2 delivery vehicle can include at least one CO2 storage device for retaining the CO2 for delivery of the CO2 to a port fuel production system CO2 storage device 21v. The releasable connection of the CO2 delivery conduit 21s with the CO2 storage device(s) 6s can permit CO2 delivery to occur via different ships at different times using the same CO2 delivery conduit 21s or CO2 delivery conduit arrangement.

For example, the CO2 storage device(s) 6s can be positioned and configured so that CO2 from within the CO2 storage device(s) can be releasably coupled to a CO2 delivery vehicle (e.g. a CO2 tanker, or other type of CO2 transport vessel). The CO2 delivery vehicle can receive the CO2 from the CO2 storage device(s) 6s via a CO2 delivery conduit 21s that can be releasably connected between the CO2 delivery vehicle and the CO2 storage device(s) 6s while the ship is in water and not docked at a port. The CO2 delivery vehicle can then convey the captured CO2 to a port fuel production system (PFPS) 21 for the PFPS 21 to utilize the captured CO2. In other embodiments, the ship 2 can provide the CO2 more directly to the PFPS 21 via the CO2 delivery conduit 21s for supplying CO2 from the CO2 storage device(s) 6s that are on the ship 2 to the port fuel production system CO2 storage device 21v.

The port fuel production system (PFPS) 21 can be on site at a port near where the ship 2 can be docked or where a CO2 delivery vehicle can be docked. The PFPS can include at least one PFPS CO2 storage tank 21v that can be fluidly connectable to the CO2 storage device(s) 6s of one or more ships 2 for receipt of CO2 captured by the at least one CCS 6 of the ship(s) 2. The CO2 can be fed to the CO2 storage tank 21v as a liquid or a gas. In some embodiments, a pump, blower, or compressor may be utilized to help facilitate the flow of the CO2 from the storage device(s) 6s to the PFPS CO2 storage tank(s) 21v. An expander can be used to recover energy from the liquid CO2 as it expands to gas prior to being feed to the PFPS or before being fed to a reactor of the fuel production system 21p.

The PFPS 21 can also include a source of hydrogen 21h. The source of hydrogen (H2) can be an H2 source such as an H2 pipeline or at least one storage tank that stores H2 therein. The stored hydrogen can be hydrogen received from at least one hydrogen manufacturing facility. In some embodiments, the hydrogen that is utilized can be hydrogen formed via utilization of renewable power (e.g. electrolysis of water powered by renewable energy sources) or hydrogen that is formed via use of more conventional processing that may utilize carbon capture or other technologies to help limit CO2 emissions associated with the manufacture of the hydrogen (e.g. blue hydrogen). In yet other embodiments, the hydrogen may be from a more conventional hydrogen manufacturing source. It can be preferred to utilize hydrogen generated from renewable power or hydrogen formed in conjunction with carbon capture technologies to help limit CO2 emissions associated with the hydrogen production in embodiments in which it is desired to help keep CO2 emissions associated with the hydrogen (H2) as low as possible.

The source of hydrogen 21h and the CO2 storage tank(s) 21v of the PFPS 21 can be fluidly connected to a fuel production system 21p. The fuel production system can be configured to form methanol, dimethyl ether, methane, or liquid natural gas (LNG) via the H2 from the source of H2 21h and the CO2 from the PFPS CO2 storage tank(s) 21v. In some embodiments, the PFPS 21 can utilize a methanation reactor system or other suitable methanation or methanol generation system that can utilize CO2 and H2 to form methane (e.g. natural gas, liquid natural gas, etc.), methanol or dimethyl ether. In some embodiments, a membrane system can be used downstream from the reactor to recover CO2 and H2 from the produced fuel to be recycled back to the reactor. The formed fuel (e.g. methane, methanol, dimethyl ether, etc.) can be output from the fuel production system 21p and fed to a fuel storage device 21d of the PFPS. The fuel storage device 21d can be positioned to feed fuel to one or more dispenser devices or other downstream devices for feeding the formed fuel to the fuel source 3 of the ship 2 (e.g. ship fuel tank(s), etc.). The distribution of fuel can be provided to the fuel sources of multiple different ships 2 at the same time via different dispensers in some embodiments. The PFPS fuel storage device 21d can be fluidly connectable to the ship fuel source(s) 3 via at least one ship fueling feed conduit 21f positioned between the PFPS fuel storage device 21d and the fuel source(s) 3 of the ship(s) 2. One or more pumps can be fluidly connected to the fueling feed conduit 21f to help provide the fuel to the ship(s) 2.

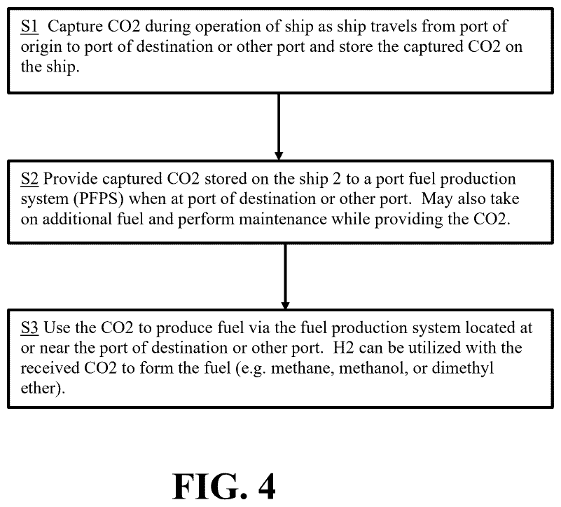

Embodiments of the apparatus 1 can be utilized in conjunction with an embodiment of a process for ship-based CO2 capture. Embodiments of such a process can be utilized in a process for ship-based CO2 capture for use in fuel production, for example. FIG. 4 illustrates an example of such a process.

In a first step S1, CO2 can be captured during operation of a ship 2 as the ship travels from a port of origin to a port of destination or other port (e.g. an intermediate port between its port of origin and port of destination). The CO2 that is captured can be CO2 captured by a CCS 6 that captures CO2 from flue gas output from an engine of the ship 2, for example. The captured CO2 can be stored in at least one CO2 storage device 6s of the ship 2. The CO2 can be stored as a liquid, as a mix of gas and liquid, or as a gas. In some configurations, the CO2 can be stored in a storage tank including a solution that may retain the CO2 therein for storage (e.g. an amine-based solution that may be used in a scrubber of an absorption-based system of the CO2 capture device 6cd, etc.)).

In a second step S2, the captured CO2 that can be stored on the ship 2 (e.g. CO2 captured from flue gas and stored in ship's CO2 storage device(s) 6s) can be provided to a port fuel production system (PFPS) at the ship's port of destination or other port that the ship 2 may stop at during its travel between its port of origin and port of destination. The ship 2 can also take on additional fuel for storage in its fuel source 3 (e.g. fuel tank(s), etc.). Other maintenance work can also be performed on the ship 2 while it is providing CO2 from its CO2 storage device(s) 6s to the CO2 storage tank(s) 21v of the PFPS via at least one CO2 delivery conduit 21s that is coupled between the ship's CO2 storage device(s) 6s and the CO2 storage tank(s) 21v.

In some embodiments, the CO2 captured and stored on the ship 2 via the CO2 storage device(s) 6s can be output to an intermediate CO2 delivery vehicle. Such a CO2 delivery vehicle can be a tanker or other type of CO2 delivery vehicle that can be positioned near the ship 2 to receive the CO2 from the CO2 storage device(s) 6s and store the CO2 therein for subsequent delivery to the PFPS 21. That intermediate delivery vehicle can provide the CO2 from the ship 2 to the CO2 storage tank(s) 21v. In other embodiments, a CO2 supply conduit 21s can be utilized to provide the CO2 to the CO2 storage tank(s) 21v of the PFPS 21.

In a third step S3, the port PFPS 21 can utilize the stored CO2 received from the ship(s) 2 for producing fuel via a fuel production system 21p of the PFPS. In some embodiments in which the CO2 may be captured via a solution, the solution (e.g. an amine-based solution) may be passed through a stripper for the PFPS to release the CO2 for feeding to the fuel production system. In other embodiments in which the CO2 may be stored in liquid or gas form, the CO2 can be more directly stored and fed to the fuel production system 21p.

The fuel production system 21p can also utilize hydrogen (H2) from at least one source of H2 21h to form the fuel. The formed fuel can be methane, methanol, or DME, for example.

The process can also include other steps or features. For example, the produced fuel can be fed to a fuel storage device 21d of the PFPS. The fuel stored in the fuel storage device 21d of the PFPS can be fed to one or more ship fuel tanks or other source of fuel 3 for the ship(s) 2 via at least one ship fueling feed conduit 21f that can be positioned between the fuel storage device(s) 21d of the PFPS and the source(s) of fuel 3 for the ship(s) 2. The process can also include other steps or features associated with purification of the formed fuel or purification of the H2 or CO2 for use in forming the fuel. In yet other embodiments, the process can also include other steps or features.

Embodiments of the process and apparatus 1 can permit decentralized capture of CO2 during operations of various different ships to occur to limit CO2 emissions of the ships 2 and also permit that captured CO2 to be utilized at different ports for production of fuel in a way that can efficiently utilize the captured CO2 for fuel production while also limiting CO2 emissions associated with the fuel production and the operation of the ships 2. Further, embodiments can permit the operation of the ships 2 to result in formation of a useful product (e.g. CO2) that can be sold to a PFPS to help enhance the profitability in operation of the ships 2. Embodiments can permit CO2 capture to occur without needing to sequester the CO2 in geological formations or other masses so that the captured CO2 provides a benefit to a ship operator to help financially encourage the reduction of CO2 emissions. Embodiments can also be provided to negate the need to transport CO2 via pipeline or other means to other remote storage locations.

Further, embodiments can permit operations of port fuel production systems to have enhanced flexibility by providing an additional source of CO2 for receipt of CO2 for use in operations to form fuel. This flexibility can be provided while also providing the CO2 in a more ecologically sustainable process.

It should also be appreciated that modifications to embodiments of the apparatus 1 and process can also be made to meet a particular set of criteria for different embodiments of the apparatus or process. For instance, the arrangement of valves, sensors, piping, and other conduit elements (e.g., conduit connection mechanisms, tubing, seals, valves, etc.) for interconnecting different units of the apparatus for fluid communication of the flows of fluid between different elements can be arranged to meet a particular facility layout design that accounts for available area of the apparatus, sized equipment of the apparatus, a preferred type of automated process control scheme and/or distributed control scheme, and other design considerations. The size or type of the equipment can be modified to meet a particular set of design criteria as well.

As yet another example, it is contemplated that a particular feature described, either individually or as part of an embodiment, can be combined with other individually described features, or parts of other embodiments. The elements and acts of the various embodiments described herein can therefore be combined to provide further embodiments. Thus, while certain exemplary embodiments of the process, apparatus, system, and methods of making and using the same have been shown and described above, it is to be distinctly understood that the invention is not limited thereto but may be otherwise variously embodied and practiced within the scope of the following claims.

1. An apparatus for ship-based carbon dioxide (CO2) capture, comprising:
a ship having a CO2 capture system positioned to receive flue gas from an engine of the ship to remove CO2 from the flue gas; the CO2 capture system configured so that CO2 removed from the flue gas is storable in at least one CO2 storage device of the ship that is in fluid communication with a CO2 capture device of the CO2 capture system of the ship;
the CO2 storage device of the ship being releaseably connectable to a CO2 delivery conduit connectable to the CO2 storage device to feed CO2 from the CO2 storage device of the ship to at least one CO2 storage tank of a port fuel production system (PFPS) positioned at a port in which the ship is dockable or a CO2 delivery vehicle positionable adjacent the ship to receive the CO2 from the CO2 storage device of the ship to the delivery vehicle for supplying the CO2 to the PFPS.

2. The apparatus of claim 1, wherein the apparatus includes the PFPS and the PFPS include a fuel production system configured to form methane, dimethyl ether (DME), or methanol from the CO2 provided by the CO2 storage device of the ship and hydrogen from at least one source of hydrogen.

3. The apparatus of claim 2, wherein the fuel production system includes a methanation reactor and/or a methanation and membrane system.

4. The apparatus of claim 2, wherein the PFPS includes a fuel storage device that is positioned to receive the fuel formed via the fuel production system, the fuel storage device being connectable to a source of fuel of the ship via a ship fueling feed conduit connectable between the source of fuel of the ship and the fuel storage device of the PFPS.

5. The apparatus of claim 4, wherein the fuel comprises methane, dimethyl ether (DME), or methanol.

6. The apparatus of claim 2, wherein the source of hydrogen is a hydrogen pipeline or a hydrogen storage tank.

7. The apparatus of claim 2, wherein the source of hydrogen is hydrogen formed via electrolysis of water.

8. The apparatus of claim 7, wherein the electrolysis of water is powered by renewable power.

9. The apparatus of claim 5, wherein the source of hydrogen is hydrogen manufactured via a blue hydrogen production process.

10. A process for ship-based carbon dioxide (CO2) capture, comprising:
capturing CO2 from flue gas emitted by an engine of a ship during operation of the ship via a carbon capture system of the ship,
storing the captured CO2 in at least one CO2 storage device of the ship that is in fluid communication with a CO2 capture device of the CO2 capture system of the ship for subsequent delivery of the CO2 stored in the CO2 storage device of the ship to (i) a CO2 delivery conduit connectable to the CO2 storage device to feed CO2 from the CO2 storage device of the ship to at least one CO2 storage tank of a port fuel production system (PFPS) positioned at a port in which the ship is dockable or (ii) a CO2 delivery vehicle positionable adjacent the ship to receive the captured CO2 from the CO2 storage device of the ship to the delivery vehicle for supplying the CO2 to at least one CO2 storage tank of the PFPS.

11. The process of claim 10, comprising:
docking the ship at the port;
connecting the CO2 storage device of the ship to the CO2 delivery conduit to feed the CO2 stored in the CO2 storage device of the ship to the at least one CO2 storage tank of the PFPS; and
feeding the CO2 from the CO2 storage device of the ship to the at least one CO2 storage tank of the PFPS via the CO2 delivery conduit.

12. The process of claim 11, comprising:
feeding the CO2 from the at least one CO2 storage tank of the PFPS to a fuel production system of the PFPS to form a fuel; and
feeding hydrogen from a source of hydrogen of the PFPS to the fuel production system to form the fuel.

13. The process of claim 12, comprising:
feeding the formed fuel from the fuel production system to a fuel storage device of the PFPS.

14. The process of claim 13, comprising:
feeding the fuel stored in the fuel storage device of the PFPS to a source of fuel of the ship.

15. The process of claim 14, wherein the feeding of the fuel to the source of fuel of the ship occurs at a same time the feeding of the CO2 from the CO2 storage device of the ship to the at least one CO2 storage tank of the PFPS via the CO2 delivery conduit occurs.

16. The process of claim 15, wherein the fuel is comprised of methane, dimethyl ether (DME), or methanol.

17. A system for ship-based carbon dioxide (CO2) capture, comprising:
a ship having a CO2 capture system positioned to receive flue gas from an engine of the ship to remove CO2 from the flue gas; the CO2 capture system configured so that CO2 removed from the flue gas is storable in at least one CO2 storage device of the ship that is in fluid communication with a CO2 capture device of the CO2 capture system of the ship;
the CO2 storage device of the ship being releaseably connectable to a CO2 delivery conduit connectable to the CO2 storage device to feed CO2 from the CO2 storage device of the ship to at least one CO2 storage tank of a port fuel production system (PFPS) positioned at a port in which the ship is dockable; and
the PFPS having a source of hydrogen and a fuel production system that is fluidly connectable to the source of hydrogen and the at least one CO2 storage tank of the PFPS so that a fuel is producible via the fuel production system via use of the CO2 from the at least one CO2 storage tank and hydrogen from the source of hydrogen.

18. The system of claim 17, wherein the source of hydrogen includes hydrogen produced via electrolysis of water powered by renewable power or hydrogen produced via a blue hydrogen production process.

19. The system of claim 17, wherein the fuel is comprised of methane, dimethyl ether (DME), or methanol.

20. The system of claim 17, wherein the PFPS has a fuel storage device connected to the fuel production system to receive and store the fuel output from the fuel production system, the fuel storage device of the PFPS being fluidly connectable to a source of fuel of the ship via a ship fueling feed conduit connectable between the source of fuel of the ship and the fuel storage device of the PFPS.
